(19) United States
(12) Patent Application Publication
(10) Pub. No.: US 2015/0306225 A1
(43) Pub. Date: Oct. 29, 2015

(54) Compositions for Delivering Drugs with Low Water Solubility

(71) Applicant: Juntech Pharmaceuticals L.L.C., Ballwin, MO (US)

(72) Inventor: Jun Yang, Ballwin, MO (US)

(21) Appl. No.: 14/662,313

(22) Filed: Mar. 19, 2015

Related U.S. Application Data
(60) Provisional application No. 61/955,291, filed on Mar. 19, 2014.

(51) Int. Cl. A61K 47/28; A61K 9/107; A61K 31/436; A61K 31/517; A61K 31/7048; A61K 31/337; A61K 38/13

(52) U.S. Cl. CPC A61K 47/28; A61K 31/337; A61K 9/1075; A61K 38/13; A61K 31/517; A61K 31/7048; A61K 31/436

(57) ABSTRACT

The present invention provides compositions comprising polymeric micelles containing a drug with low water solubility, wherein sterol rings of pegylated sterols and the drug form a hydrophobic portion and polyethylene glycol chains of the pegylated sterols forms a hydrophilic portion. The present invention also provides methods of preparing and using such compositions for delivering drug(s) with low water solubility to a subject in need thereof.

### CROSS-REFERENCE TO RELATED APPLICATIONS

This U.S. Non-Provisional Patent Application claims the benefit of U.S. Provisional Patent Application No. 61/955,291, filed Mar. 19, 2014 and incorporated herein by reference in its entirety.

### FIELD OF THE INVENTION

The present invention generally relates to the fields of drug delivery and medical or veterinary treatment. In particular, the present invention relates to compositions for delivering drugs with low water solubility in aqueous medium without cosolvent or surfactant. The present invention also relates to methods of making and using such compositions for medical or veterinary treatment.

### BACKGROUND OF THE INVENTION

Delivering drugs with low water solubility is a challenging task for pharmaceutical industry especially for those drugs that need to be administrated by intravenous injection [1]. The hydrophobic nature of those drugs makes it difficult to prepare aqueous formulation with desired level of therapeutics. Solvents and surfactants are therefore frequently employed in such type of formulations to enhance the drug solubility in aqueous medium. For example, Cremophor EL and ethanol are used for formulations such as paclitaxel (Taxol®) [2], ixabepilone (Ixempra®) [3], Cyclosphorine (Sandimmune®) [4], and teniposide (Vumon®) [5] while polysorbate 80 and ethanol are used for formulations such as docetaxel formulation (Taxotere®) [6] and Etoposide (Toposar®) [7]. However, the intrinsic toxicity associated with these surfactants and solvents limit their usage and premedication may be required to relief the side effects. For example, Cremophor® EL (polyoxyethylated castor oil) is associated with anaphylactic reactions in some patients that received Sandimmune® Injection (cyclosporine injection, USP) [4]. The side effects include flushing of face and upper thorax, noncardiogenic pulmonary edema, acute respiratory distress, dyspnea, wheezing, blood pressure changes, and tachycardia. These types of anaphylactic reactions, however, have not been reported with the soft gelatin capsules or oral solution which lacks Cremophor® EL in the formulation. Another typical example is Taxol® formulation where the Cremophor® is found to be associated with acute hypersensitive reaction in some patients. Premedication with steroids and antihistamines are therefore required to reduce the risk of such reactions. Even so, these types of hypersensitivity reactions are not always eliminated and sometimes lead to fatalities [8]. Similar hypersensitive reactions were also reported for patients medicated with other drugs containing Cremophor® EL and polysobate 80. Therefore, there are still unmet needs to develop alternative intravenous formulations for drugs with low water solubility without using surfactants and solvents.

The use of polymeric micelle as a drug delivery carrier has become one of intensively studied area for drugs with low water solubility. Genexol-PM® [9] is a polymeric micelle comprising monomethyoxy-PEG-b-poly(D,L-lactide) (MPEG-PDLLA) developed by Samyang of Korea. The hydrophilic segment is PEG with molecular weight of 2000 g/mol and the hydrophobic segment is poly(D,L-lactide) with molecular weight of 1200 g/mol. The self-assembled polymeric micelle has a particle size of 20-50 nm and a drug loading efficiency of 16.7% by weight. Related preclinical studies indicate that the MPEG-PDLLA polymeric micelle is non-toxic and biocompatible both in vitro and in vivo. Compared to Taxol®, Genexol-PM® showed a significant decrease in Area Under Curve (AUC) after intravenous administration when given in equivalent dose, which may be contributed by dissociation of MPEG-PDLLA micelles in the presence of α and β-globulin in the blood, resulting in a rapid release of paclitaxel from the micelles. Despite the decrease in AUC, the subsequent clinical studies demonstrated a superior efficacy and lower toxicity for Genexol-PM® which led to its approval of use in several countries.

NK105 is another polymeric micelle formulation comprising PEG-poly(aspartic acid) modified with 4-phenyl-1-butanol developed by Nanocarrier in Japan [10]. The hydrophilic segment was PEG with molecular weight of 8000 and the hydrophobic segment was poly(aspartic acid) with 4-phenyl-1-butanol modified with molecular weight of 20,000. The average particle size of the micelles is approximately 85 nm with a wide range of size distributions from 20 to 430 nm. Drug loading efficiency is about 23% by weight. Related clinical trials indicate that NK105 has reduced toxicity compared to Taxol® formulation. Increased AUC and decreased total clearance were also observed for NK105 formulation compared to Genexol-PM® at 300 mg/m², which suggests higher blood stability of NK105. Clinical trials are currently conducted for NK105.

For the above-mentioned two types of polymeric micelles, PEG is the hydrophilic segment while the hydrophobic segment is either lactic acid or aspartic acid. Although the polymerized lactic acid (PLA) and polymerized aspartic acid (PAA) are hydrophobic in nature, they are linear molecules which may lack affinity for phenyl structure hydrophobic material such as taxanes or podophyllotoxins. From a formulation point of view, the hydrophobic interactions between drug molecules and these polymers may not be strong enough to provide a stable intravenous injection formulation. Thus, there is still a need for a stable polymeric micelle formulation for delivering drugs with low water solubility such as taxanes, podophyllotoxins or other aromatic ring structure-containing drugs. The present invention fulfills such a need.

### SUMMARY OF THE INVENTION

Provided herein are unique polymeric micelle based compositions for delivering pharmaceutics with low water solubility in aqueous medium, methods of preparing such compositions, and methods of using such compositions for delivering drugs with low water solubility to in-need subjects by means of oral, parenteral, topical, transdermal or transmucosal administration.

In one embodiment, there is provided a composition comprising polymeric micelles containing a drug with low water solubility, wherein sterol rings of pegylated sterols and the drug form a hydrophobic portion and polyethylene glycol chains of the pegylated sterols forms a hydrophilic portion.

In another embodiment, there is provided a method of preparing a pharmaceutical or veterinary composition comprising polymeric micelles containing a drug with low water solubility. Such method comprises mixing the drug with at least one pegylated sterol, or derivative thereof, and then subjecting the mixture to solvent evaporation, dialysis, ultrasonification, stirring, or any combination thereof to form polymeric micelles containing the drug.

In still another embodiment, there is provided a method for delivering a drug with low water solubility to a subject in need thereof. Such method comprises administering a therapeutically effective dose of the composition of the present invention to the subject.

Other embodiments, features, and advantages of the compositions and methods provided herein will be apparent from the following detailed description, examples, and claims.

### BRIEF DESCRIPTION OF THE DRAWINGS

FIG. 1 illustrates release profile of paclitaxel formulations at 37° C. using 1.0 M sodium salicylate as medium for release test.

FIG. 2 illustrates release profile of docetaxel formulations at 37° C. using 1.0 M sodium salicylate as medium for release test.

### DETAILED DESCRIPTION OF THE INVENTION

Provided herein are pharmaceutical and veterinary compositions that comprise polymeric micelles containing a drug with low water solubility. The polymeric micelles act as a carrier to deliver the drug in aqueous medium without cosolvent or surfactant, wherein hydrophobic portion of the amphiphilic polymer formed by sterol rings of pegylated sterols imparts hydrophobic interaction to retain the drug inside the micelles, and the hydrophilic portion of the amphiphilic polymer formed by polyethylene glycol chains of the pegylated sterols provides the hydrophilic shell to keep the micelles stable in aqueous medium.

In one embodiment, the composition of the present invention comprises from about 60% to about 99.99% by molar percent of the polymeric micelles and from about 0.01% to about 40% by molar percent of the drug. The composition may further comprise an excipient selected from the group consisting of a diluent, a binder, a disintegrant, a lubricant, and a colorant.

In one embodiment, the sterol can be an animal, plant, fungal, and/or algal sterol, and the pegylated forms of these sterols comprise polyethylene glycol molecules with an average molecular weight in the range of 1000 to 5000 daltons. Exemplary pegylated sterols that are suitable for the present invention include, but are not limited to, pegylated cholesterol, pegylated sitosterol, pegylated campesterol, pegylated stigmasterol, pegylated campestanol, pegylated brassicasterol, pegylated lathosterol, pegylated ergosterol, pegylated cycloartenol, pegylated cyclolaudenol, pegylated protothecasterol, pegylated 4a-methylergostanol, pegylated 4a-methylclionastanol, pegylated clionastanol, pegylated 24-beta-ethylcholesta-8,22-enol, derivatives thereof, and a combination thereof.

In the polymeric micelle composition of the present invention, the connected ring structure of pegylated sterols provides hydrophobic interactions with hydrophobic drugs. The drug encapsulation efficiency of the polymeric micelles is dependent on the affinity between the hydrophobic segment/portion of the polymers and the drug molecule. A general indication of good compatibility is the structural similarity between the drug molecules and hydrophobic segment/portion. Similar to the “like dissolves like” rule, the pegylated sterol polymeric micelles will be more compatible for drug molecules with benzyl ring structures than linear polymers such as Poly(lactic acid) or Poly(aspartic acid). Flory-Huggins interaction parameter, Xsp, is a commonly used parameter to quantify the interaction[11]:

$${X}_{sp} = \frac{{( δ s - δ p )}^{2} {V}_{s}}{kT}$$

Where δs and δp are the solubility parameters for drug and the hydrophobic segment respectively, Vs is the molar volume of the drug, k is the Boltzmann constant, and T is the temperature in Kelvin. Theoretically, minimization of Xsp leads to better compatibility for high drug loading efficiency although many other factors should also be considered. For example, the length of hydrophilic segment (molecular weight of PEG) also plays an important role for drug loading efficiency. Suitable polymeric micelle for a drug with low water solubility can only be obtained by screening different combinations of hydrophobic segment and hydrophilic segment systematically.

In certain embodiments, compositions provided herein are based on sterol ring structure such as cholesterol or phytosterol structure which comprises four rings. Without seeking to be limited by theory, it is postulated that the similarity between the sterol structure and aromatic or other hydrophobic ring-containing hydrophobic drugs make it possible to form a polymeric micelle with better affinity and therefore with a high loading efficiency. Additionally, cholesterol is an important ingredient for human cell membrane and is compatible in plasma thus safe to use for constructing the polymeric micelles. Similarly, phytosterols have been used for food and dietary supplements for decades and therefore are also safe components for the polymeric micelles.

Regular PEG molecules can be employed as the hydrophilic segment/portion of the polymeric micelles because they are non-toxic and have been approved by FDA for drug applications. Additionally, pegylation is well established as stealth agent to reduce reticuloendothelial system (RES) uptake to ensure long time circulation [12]. Moreover, the functional groups of PEG make it easy to conjugate with sterol molecules such as cholesterol.

In one non-limiting example, a polymeric micelle comprising pegylated cholesterol is provided herein for delivering drugs with low water solubility.

In general, it is believed that a wide variety of pegylated sterols can be used to form the polymeric micelles containing hydrophobic drugs. Without seeking to be limited by theory, it is believed that the hydrophobic drugs are physically trapped within the inner core region of the micelles that contains the sterol rings of the pegylated sterols and non-covalently bonded through hydrophobic interactions. In certain embodiments, the hydrophilic PEG segment of the polymeric micelles provides colloidal stability in aqueous medium and provides stealth function for long time circulation in plasma. In certain embodiments, the polymeric micelle is characterized with a diameter of 10 to 30 nm (nanosized) and a polydispersity index of less than 0.1. In certain embodiments, the narrowly distributed polymeric micelle is in a transparent aqueous solution in which the hydrophobic drug is physically trapped. In certain embodiments, the compositions provided herein are suitable for preparing parenteral injections of taxanes, podophyllotoxins or other benzene ring-containing hydrophobic drugs. In certain embodiments, the compositions provided herein are suitable for preparing oral, parenteral, or other dose forms of hydrophobic drugs that comprise hydrophobic rings and/or aromatic rings that are hydrophobic.

The amphiphilic polymer used for the present invention comprises two functional groups: a hydrophobic group based on cholesterol or phytosteol or derivative thereof and a hydrophilic group based on PEG. The ether, ester or other similar connections could be generated between the two functional groups. The hydrophobic group has a molecular weight of more than 386.7 daltons and the PEG group has a molecular weight of more than 1000 daltons, preferably 1000 to 5000 daltons. Without limiting the scope of the present invention, the amphiphilic polymer could be any type of conjugated polymers comprising the two functional groups as described above which could be readily synthesized by those skilled in the art of synthetic organic chemistry.

The drugs with low water solubility which are suitable for delivering by the polymeric micelles provided herein can be any bioactive agent having limited solubility in aqueous medium. In one embodiment, the drugs with low water solubility comprise an aromatic ring structure selected from the group consisting of a benzyl ring, a pentadiene ring, a thiophene ring, and a furan ring. In certain embodiments, the drugs with low water solubility can comprise a hydrophobic ring. In certain embodiments, such hydrophobic rings can be a 3-, 4-, 5-, 6-, 7-, 8-, or more carbon rings that can be saturated, unsaturated, substituted, or unsubstituted. Hydrophobic drugs used in the compositions and methods provided herein include, but are not limited to, an anticancer drug, an anti-fungal drug, an anti-viral drug, an anti-bacterial drug, an immune-suppressant, a tyrosine kinase inhibitor, an antiphlogistic anodyne, a hormone composition, an anti-allergy drug, a hepatism drug, a metabolic drug, a drug for treating a central nervous system disease, a drug for treating a respiratory disease, a drug for treating a peripheral disease, a drug for treating a digestive disease, a drug for treating an infectious disease, or a drug for treating a circulatory disease. Exemplary hydrophobic drugs used in the compositions and methods provided herein include, but are not limited to, paclitaxel, docetaxel, cyclosporine, teniposide, etoposide, doxorubicin, daunomycin, mitomycin C, sirolimus, everolimus, indomethacin, ibuprofen, lapatinib ditosylate, and biphenyl dimethyl dicarboxylate. In certain embodiments, paclitaxel, docetaxel, cyclosporine, teniposide, lapatinib ditosylate, sirolimus and everolimus are used in the compositions and methods provided herein.

Also provided herein are methods of preparing a pharmaceutical or veterinary composition that comprises polymeric micelles containing a drug with low water solubility. In certain embodiments, such methods can comprise mixing the drug with at least one pegylated sterol, or derivative thereof, and then subjecting the mixture to solvent evaporation, dialysis, ultrasonification, stirring, or any combination thereof to form polymeric micelles containing the drug.

In particular, one or more of the drugs with low water solubility can be incorporated into polymeric micelles by using various methods. One of the methods is solvent evaporation and rehydration method, which comprises the steps of (a) dissolving the drug(s) and the pegylated sterol into a water-immiscible organic solvent to form a homogenous mixture; (b) removing the organic solvent by slowly evaporating it to form a film; and (c) rehydrating the film by aqueous medium to form polymeric micelles. Another method is dialysis method, which comprises the steps of (a) dissolving the drug(s) and the pegylated sterol into a water-immiscible organic solvent to form a homogenous mixture; and (b) dialyzing the mixture against a buffer solution and then against water. Still another method is through ultrasonification, which comprises the steps of (a) dissolving the pegylated sterol into an aqueous solution; (b) adding the hydrophobic drug(s) into the solution; (c) ultrasonificating the solution for about 1 second to about 1 hour; and (d) stirring the mixture to obtain a polymeric micelle formulation. Still another method is through stirring, which comprises the steps of (a) preparing an aqueous solution of polymeric micelles; and (b) adding the hydrophobic drug(s) to the prepared solution; and (c) stirring for a period of time until all drug(s) are dissolved to form a clear solution. Combinations of two or more of the above methods can also be used for preparing pharmaceutical or veterinary compositions provided herein

For all the methods mentioned above, lyophilization of polymeric micelle drug solution can optionally be followed after the preparation to extend storage stability. Sucrose, maltose, mannitol or other lyophilization aids could be employed to facilitate lyophilization and reconstitution process. Spray drying or similar drying techniques can also be employed after preparing the polymeric micelle solution to form dry powders. The dried polymeric micelle powders can be self-emulsified in gastrointestinal tract for delivering hydrophobic drugs orally. Topical, transdermal or transmucosal forms of the composition can also be prepared using appropriate formulation techniques.

In certain embodiments, polymeric micelles provided herein can have a diameter of 10 to 30 nm with a narrow particle size distribution (PDI<0.1). In certain embodiments, polymeric micelles are in stable and transparent aqueous solutions suitable for intravenous injection. Due to the strong hydrophobic interactions between the sterol rings of pegylated sterols and the hydrophobic drug, therapeutical concentration of such composition could be achieved without involving other solvents or surfactants. For example, water solubility of paclitaxel is about 0.3 μg/ml. Due to such low water solubility, paclitaxel is formulated with Cremophor EL along with ethanol conventionally. In Taxol® formulation, each mL of sterile nonpyrogenic solution contains 6 mg paclitaxel, 527 mg of purified Cremophor® EL (polyoxyethylated castor oil) and 49.7% (v/v) dehydrated alcohol. As recommended by the package inserts of Taxol®, the dose for Taxol® is 175 mg/m² for about 3 to 24 hours infusion. Therefore, a total dose of 300 mg of paclitaxel is administrated to an adult patient in each cycle of treatment, which corresponds to a total of 26.35 g of Cremophor® EL and 25 mL of ethanol. Practically, Taxol® will be diluted to 0.3 to 1.2 mg/ml in isotonic saline or 5% dextrose solution before injection. The diluted formulation is physically and chemically stable for up to 27 hours at room temperature and may form precipitates which require the use of a filter in the injection line. In addition, a relatively long period of infusion time (about 3 to 24 hours) is required to reduce the potential hypersensitive reaction caused by Cremophor® EL in Taxol® formulation.

In contrast, the polymeric micelles provided herein can load up to 10.0% (by molar) (vs. Taxol of 2.14%) of paclitaxel in the formulation (as illustrated in Example 1 below). The high loading efficiency of paclitaxel in the composition provided herein result in a substantial reduction of the amount of amphiphilic polymer in the composition. Given the same dose of paclitaxel, only 5.7 grams of pegylated-cholesterol will be administrated without ethanol. When the polymeric micelle formulation subjected to dilution for reconstitution, the formed micelles have a size range of 10 to 30 nm with narrow size distribution. The diluted polymeric micelles are stable both physically and chemically for over 48 hours in room temperature without size change as demonstrated by Malvern particle size measurements. Furthermore, the in vitro release test results demonstrate the composition of the present invention has a slow release profile that is comparable to Taxol® at similar conditions. The exemplary results presented below all suggest that the polymeric micelle compositions provided herein have improved safety and higher efficacy profiles compared to conventional formulations.

In preparing the pharmaceutical or veterinary composition of the present invention, drug loading efficiency has to be taken into consideration. Hydrophobic interactions between the drug molecules and the hydrophobic portion of the amphiphilic polymer (i.e., sterol rings of pegylated sterols) and the molecular weight of hydrophilic PEG segment all influence the drug loading efficiency. Therefore, the balance between the hydrophobic segment and hydrophilic segment will need to be optimized to reach the maximum loading efficiency. It is generally anticipated that in certain embodiments the drug with low water solubility will make up from about 0.01% to about 40% by molar percent of the polymeric micelle composition. In certain embodiments, the drug with low water solubility will make up from about 1% to about 20% by molar percent of the polymeric micelle composition. However, these ranges are not meant to limit the scope of the invention.

Also provided are methods for delivering a drug with low water solubility to a subject in need thereof. In certain embodiments, such methods comprise administering the composition of the present invention to the subject. Subjects of the methods provided herein include both human and veterinary subjects. Veterinary subjects include, but are not limited to, cattle, horses, pigs, goats, chickens, and companion animals such as dogs and cats. In one embodiment, the subject suffers from a disease/condition selected from the group consisting of a cancer, an immune system disorder, an allergy, a liver disorder, a metabolic disorder, a central nervous system disease, a respiratory disease, a peripheral disease, a digestive disease, an infectious disease, and a circulatory disease. In one embodiment, the composition may be administrated by oral, parenteral, topical, transdermal, transmucosal or intrathecal means.

In summary, the present invention provides a polymeric micelle composition for delivering drugs with low water solubility with higher efficacy and safety profiles compared to traditional formulations as well as methods of preparing and using such composition.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. It should be appreciated by those of ordinary skill in the art that the techniques disclosed in the examples that follow represent approaches the inventors have found function well in the practice of the invention, and thus can be considered to constitute examples of preferred modes for its practice. However, those of ordinary skill in the art should, in light of the present disclosure, appreciate that many changes can be made in the specific embodiments that are disclosed and still obtain a like or similar result without departing from the spirit and scope of the invention.

### Example 1

### Preparing a Polymeric Micelle Containing Paclitaxel

252 mg of paclitaxel (LC laboratory) and 4753 mg of CS-20 (NOF) were dissolved into a round bottom flask with 50 mL of absolute ethanol (Fisher) at room temperature. The solution was rota-evaporated at 65° C. for about 1 hour to remove solvent. The film formed in round bottom flask was further vacuumed overnight at room temperature in the hood. The obtained dry film was rehydrated with 50.0 mL of 300 mM sucrose (Fisher) at 45° C. for about 60 minutes. The rehydrated solution was clear and free of visible particles. The solution was cooled down to room temperature and then filtered through a 0.22 um filter (Whatman). The filtered solution was collected in a 100 mL serum vial and labeled as E1120. Size characterization of E1120 was conducted by Malvern Zetasizer with 0.9% saline as dilution medium (see Table 1).

TABLE 1
Size Characterization of Paclitaxel Formulation

| Size Measurement | Test 1 | Test 2 | Test 3 |
|---|---|---|---|
| Z-average diameter (nm) | 13.59 | 13.39 | 13.15 |
| Polydispersity index (PDI) | 0.028 | 0.034 | 0.047 |

The above results show that the polymeric micelles had a size of about 13-14 nm and a PDI<0.1. The particle size distribution of rehydrated polymeric micelle solution was retested after a month of storage at refrigerated condition (e.g., at about 2° C. to 8° C.) and no size change was observed. To further increase the storage stability of rehydrated polymeric micelle solution, lyophilization was applied. The condenser temperature of lyophilizer was set under −80° C. centigrade and the vacuum was set lower than 100 mT. The vial was removed from the lyophilyzer after 4 days of lyophilization and the white powders were obtained in all vials.

The lyophilized white powders of E1120 were reconstituted by either Mill Q water or isotonic 0.9% saline and all powders were soluble in five minutes by gentle shaking. No particles were visible in the reconstituted solution. Size characterization of reconstituted E1120 indicated no particle size and distribution changes occurred. The reconstituted solution remained stable for more than 48 hours in room temperature or for more than 2 weeks under refrigerator storage conditions. Characterization of paclitaxel was performed by HPLC and the results indicated that 25.44 μg of paclitaxel was contained per mg of lyophilized powder. Characterization of CS-20 was also carried out by using HPLC and the results indicated 0.496 mg of CS-20 contained in every mg of lyophilized powder.

Based on the analytical results, the molar percentage of loaded paclitaxel in the polymeric micelle formulation of the present invention (i.e., lyophilized E1120 powder) is about 10%. The loading capacity of paclitaxel in the present study is about 5 times greater than that of Cremophor® EL formulation, wherein only 2.14% of molar percentage of paclitaxel is being loaded. Comparison of the traditional Taxol® formulation and the formulation of the present invention is summarized in Table 2.

TABLE 2
Comparison of Taxol and Paclitaxel Formulation

| | | | | | Molar ratio |
|---|---|---|---|---|---|
| Brand | Dosage | | | Amount | of API to |
| Name | forms | Formulation | Mw. | (mg/ml) | excipient |
| Taxol ® | Injectable | Paclitaxel | 853.9 | 6.0 | 2.13% |
| | solution | Cremophor | 1630 | 527.0 | |
| | | EL | | | |
| | | Ethanol | 46 | 393.0 | |
| New | Lyophilized | Paclitaxel | 853.9 | 25.4 | 9.93% |
| paclitaxel | powder | CS-20 | 1836 | 496.1 | |

### Example 2

### Preparing a polymeric micelle containing Docetaxel

251.2 mg of docetaxel (Phyton Biotech) and 4750 mg of CS-20 (NOF) were dissolved into a round bottom flask with 50 mL of absolute ethanol (Fisher) at room temperature. The solution was rota-evaporated at 65° C. for and further vacuumed overnight at room temperature to remove solvent. The same rehydration procedure as described in Example 1 was used herein for docetaxel polymeric micelles. The final clear filtered solution was collected in a 100 mL serum vial and labeled as E1126. The particle size of E1126 was measured by dilution of 100 μl of E1126 to 900 μl of 0.9% saline.

The results show the obtained polymeric micelles had a size of around 13-14 nm and a PDI<0.1. 2.0 ml of the solution was allocated to a 5 mL serum vial and stored at refrigerator. The stored sample was retested after a month of storage. Corresponding particle size results illustrated below suggest no changes on the particle size were observed. The rest of samples were subjected to lyophilization for about 48 hours and the white powders were obtained.

The lyophilized white powders of E1126 were reconstituted by either Millipore MilliQ™ filtered water or isotonic 0.9% saline and all powders were soluble in about 5 minutes by gentle shaking. No visible particles were detected in the reconstituted solution. Size characterization of reconstituted E1126 indicated no particle size and distribution changes occurred. The reconstituted solution remained stable for more than 48 hours in room temperature or for more than 2 weeks under refrigerator storage conditions.

Characterization of docetaxel was performed by HPLC and the results indicated 23.98 mg of docetaxel per mg of lyophilized powder. Characterization of CS-20 was also carried out by using HPLC and the results indicated 0.496 mg of CS-20 contained for every mg of lyophilized E1126 powder.

Based on the analytical results, the final lyophilized E1126 powder loaded with 9.89% of molar of docetaxel, which is about 2 times greater loading capacity than polysobrate 80 formulation (5.67%). Comparison of the traditional Taxotere formulation and invented formulation is summarized in Table 3.

TABLE 3
Comparison of Taxotere ® and Docetaxel Formulation

| | | | | | Molar ratio |
|---|---|---|---|---|---|
| Brand | Dosage | | | Amount | of API to |
| Name | forms | Formulation | Mw. | (mg/ml) | excipient |
| Taxo- | Injectable | Docetaxel | 807.88 | 20.0 | 5.67% |
| tere ® | solution | Polysor- | 1310 | 540.0 | |
| | | bate 80 | | | |
| | | Ethanol | 46 | 395.0 | |
| New | Lyophilized | Docetaxel | 807.88 | 24.0 | 9.89% |
| docetaxol | powder | CS-20 | 1836 | 496.4 | |

### Example 3

### Preparing a Polymeric Micelle Containing Cyclosporine a

51 mg of Cyclosphorine (AK Scientific) and 423 mg of CS-20 (NOF) were dissolved into 10 mL 3:1 (chloroform/methanol) ethanol at room temperature. The solution was rota-evaporated at 45° C. for about 1 hour to remove solvent and further vacuumed overnight in the hood. Rehydration was conducted with 10.0 mL of 0.9% saline at 50° C. for about 30 to 60 minutes. The rehydrated solution was clear and free of visible particles. The solution was cooled down to room temperature and filtered through 0.22 um filter. The clear filtered solution was collected in a serum vial and labeled as E0625A Cyc. Size measurement conducted by Malvern Zetasizer shows that the average particle size is around 13 nm with a PDI less than 0.1. The solution was stored in refrigerator for about 6 month and no visible particles were detected in the solution. Size measurement after 6 months storage was listed in Table 4.

TABLE 4
Size Characterization of E0625A

| Size Measurement | Test 1 | Test 2 |
|---|---|---|
| Z-average diameter (nm) | 13.07 | 13.06 |
| Polydispersity index (PDI) | 0.075 | 0.079 |

Characterization of Cyclosporine A was carried out by using reverse phased HPLC with C18 column. The results show that the prepared E0625A contains 4.95 mg/ml of Cyclosphorine. Characterization of CS-20 was also performed using HPLC and the results suggest that the concentration is 39.02 mg/ml.

Compared to commercial Sandimmune® formulation, where 50 mg of Cyclosphorine was dissolved into 650 mg of Cremophor® EL (9.44% by molar ratio), whereas the polymeric micelle formulation of the present invention had a molar ratio of cyclosphorine of 16.22% which is more than 50% higher than currently available commercial formulations (see Table 5).

TABLE 5
Comparison of Sandimmune ® and Cyclosporine Formulation

| | | | | | Molar ratio |
|---|---|---|---|---|---|
| Brand | Dosage | | | Amount | of API to |
| Name | forms | Formulation | Mw. | (mg/ml) | excipient |
| Sandim- | Injectable | Cyclosporine | 1202.61 | 50.0 | 9.44% |
| mune ® | solution | Cremophor | 1630 | 650.0 | |
| | | EL | | | |
| | | Ethanol | 46 | 259.6 | |
| New | Injectable | Cyclosporine | 656.66 | 5.0 | 16.24% |
| Cyclo- | solution | CS-20 | 1836 | 39.0 | |
| sporine | | Saline | | | |

### Example 4

### Preparing a Polymeric Micelle Containing Lapatinib Ditosylate

33.4 mg of Lapatinib DitoSulfonate (LC laboratory) was mixed with 542.6 mg of CS-20 (NOF) in 10 mL ethanol at room temperature. The solution was rota-evaporated at 50° C. for about 1 hour to remove solvent. The film was continuously vacuumed overnight in the hood. Rehydration was conducted with 10.0 mL of 300 mM sucrose at 50° C. for about 30 to 60 minutes based on the protocol described in Example 1. The rehydrated yellowish solution was clear and free of visible particles. When the yellowish solution cooled down to room temperature, it was filtered through 0.22 um filter and collected in a serum vial. The sample was labeled as E1029. Size characterization shows that the average diameter of the micelles was about 14 nm with narrow size distribution. 5.0 mL (5.03 g) of rehydrated solution was aliquoted for lyophilization following the procedure as described in Example 1. The rest of filtered solution was stored at refrigerator for stability test. Lyophilization was continued for about 48 hours under the same conditions as described in Example 1.

The lyophilized yellow powders were reconstituted with 3.65 mg of 0.9% saline at room temperature by gentle shaking. All lyophilized powders were dissolved into solution within about 5 minutes without foaming. The reconstituted solution was also placed into refrigerator for stability test. After two month storage in refrigerator, both reconstituted E1029 solution and rehydrated E1029 solution were stable and free of visible particles. Corresponding size measurement suggests that they were stable and no size changes were observed.

Characterization of Lapatinib ditosylate was performed by HPLC with C18 column and the results show that the lapatinib ditosylate concentration was 4.310 mg/ml for reconstituted E1029A and 3.404 mg/ml for rehydrated E1029B, respectively. Characterization of CS-20 was also carried out by using HPLC. The results show that the CS-20 concentration was 69.76 mg/ml for E1029A and 52.16 mg/ml for E1029B, respectively. Corresponding calculation indicated the Lapatinib ditosylate concentration for reconstituted and rehydrated solutions were 10.92% and 11.46% by molar percentage. Comparisons of the Tykerb® tablet formulation and the injectable solutions of the present invention were summarized in Table 6.

TABLE 6
Comparison of Tykerb ® and Lapatinib Ditosylate Formulations

| | | | | | Molar ratio |
|---|---|---|---|---|---|
| Brand | Dosage | | | Amount | of API to |
| Name | forms | Formulation | Mw. | (mg/tab) | excipient |
| Tykerb | Tablet | Lapatinib | 943.5 | 250 | N.A |
| | | Ditosylate | | | |
| | | Monohydrate | | | |
| | | MCC | | | |
| | | Magnesium | | | |
| | | Stearate | | | |
| | | Povidone | | | |
| | | SSG | | | |
| | | Orange | | | |
| | | film Coating | | | |
| E1029A | Injectable | Lapatinib | 925.46 | 4.3 | 10.92% |
| | solution | Ditosylate | | | |
| | | CS-20 | 1836 | 69.8 | |
| | | Saline | | | |
| E1029B | Injectable | Lapatinib | 925.46 | 3.4 | 11.46% |
| | solution | Ditosylate | | | |
| | | CS-20 | 1836 | 52.2 | |
| | | Saline | | | |

### Example 5

### Preparing a Polymeric Micelle Containing Teniposide

50 mg of Teniposide (LKT laboratory) with 500 mg of CS-20 (NOF) were dissolved in 10 mL of 3:1 (chloroform/methanol) ethanol at room temperature. The solution was rota-evaporated at 45° C. for about 1 hour to remove solvent. The film was continuously vacuumed overnight in the hood. Rehydration was conducted with 10.0 mL of 0.9% saline (pH=4.0) at 50° C. for about 30 to 60 minutes. The rehydrated solution was clear and free of visible particles. When the solution cooled down to room temperature, it was filtered through 0.22 um filter and collected in a serum vial. The sample was labeled as E0626B Teniposide.

Size characterization indicated the prepared polymeric micelles had a particle size of 14 nm. HPLC characterization was carried out for teniposide with C18 column and the results indicated 0.81 mg/ml of teniposide in the sample. Corresponding CS-20 analysis indicated the concentration was 47.66 mg/ml in samples.

Compared to commercial Vumon® formulation, where 10 mg of teniposide was dissolved into 500 mg of Cremophor® EL plus additional 30 mg of benzyl alcohol and 60 mg of N,N-dimethylacetamide, the formulation of the present invention had a loading capacity of teniposide of 4.48% by molar ratio without any other additives. The molar ratio of loaded teniposide for the present formulation is about 3.8 times higher than that of commercial formulation. Comparisons of the two formulations were summarized in Table 7.

TABLE 7
Comparisons of Vumon ® and Teniposide Formulations

| | | | | | Molar ratio |
|---|---|---|---|---|---|
| Brand | Dosage | | | Amount | of API to |
| Name | forms | Formulation | Mw. | (mg/ml) | excipient |
| Vumon | Injectable | Teniposide | 656.66 | 10.0 | 1.18% |
| | solution | Cremophor | 1630 | 500.0 | |
| | | EL | | | |
| | | Benzyl | 108.14 | 30.0 | |
| | | alcohol | | | |
| | | N,N- | 87.12 | 60.0 | |
| | | dimethyl- | | | |
| | | acetamide | | | |
| | | Ethanol | 46 | 336.9 | |
| New | Injectable | Teniposide | 656.66 | 0.8 | 4.27% |
| Teniposide | solution | CS-20 | 1836 | 47.7 | |
| | | Saline | | | |

### Example 6

### Preparing a Polymeric Micelle Containing Sirolimus

28 mg of rapamycin mixed with 475 mg of CS-20 and dissolved by 3:1 (methanol/chloroform) at room temperature. The solution was rota-evaporated at 50° C. for about 1 hour to remove solvent. The film was continuously vacuumed overnight in the hood. Rehydration was conducted with 8.0 mL of 0.9% saline at 45° C. for about 30 minutes. The rehydrated solution was clear and free of visible particles. When the solution cooled down to room temperature, it was filtered through 0.22 um filter and labeled as E0410 RA. Size characterization suggests that the average size is around 13 nm with PDI<0.1.

HPLC was utilized for quantification of both rapamycin and CS-20 and the results indicated 2.965 mg/ml of rapamycin and 55.6 mg/ml of CS-20 contained in the formulation. Compare to the original Rapamune® oral solution formulation in which Phosal 50 PG mixture and the polysorbate 80 were used as solubilizers, the present formulation is simpler with higher loading capacity. A brief comparison of the two formulations is listed in Table 8.

TABLE 8
Comparisons of Rapamune ® and Sirolimus Formulations

| | | | | | Molar ratio |
|---|---|---|---|---|---|
| Brand | Dosage | | | Amount | of API to |
| Name | forms | Formulation | Mw. | (mg/ml) | excipient |
| Rapamune | Oral | Sirolimus | 914.2 | 1.0 | N.A |
| | solution | Phosal 50 | 1630 | | |
| | and tablet | PG (mixture) | | | |
| | | Polysorbate | 46 | | |
| New | Injectable | Sirolimus | 914.2 | 3.0 | 9.67% |
| Sirolimus | solution | CS-20 | 1836 | 55.6 | |
| | | Saline | | | |

### Example 7

### Preparing a Polymeric Micelle Containing Everolimus

25 mg of everolimus mixed with 475 mg of CS-20 and dissolved by 3:1 (methanol/chloroform) at room temperature. The solution was rota-evaporated at 50° C. for about 1 hour to remove solvent. The film was continuously vacuumed overnight in the hood. Rehydration was conducted with 8.0 mL of 0.9% saline at 45° C. for about 30 minutes. The rehydrated solution was clear and free of visible particles. When the solution cooled down to room temperature, it was filtered through 0.22 um filter and labeled as E0411 AF. Size characterization indicates that the average size was around 13 nm with PDI<0.1.

HPLC characterization indicated 2.4 mg/ml of everolimus and 63.4 mg/ml of CS-20 was contained in the formulation. Comparison of the commercial Afinitor® tablet formulation and the present polymeric micelle formulation is shown in Table 9.

TABLE 9
Comparison of Afinitor and Everolimus Formulations

| | | | | | Molar ratio |
|---|---|---|---|---|---|
| Brand | Dosage | | | Dose | of API to |
| Name | forms | Formulation | Mw. | (mg/tab) | excipient |
| Afin- | Tablet | Everolimus | 958.2 | 2.5, 5, | N.A |
| itor ® | | | | 7.5, 10 | |
| | | Butylated | | | |
| | | hydroxy- | | | |
| | | toluene | | | |
| | | Magnesium | | | |
| | | Stearate | | | |
| | | Lactose | | | |
| | | Monohydrate | | | |
| | | Hypromellose | | | |
| | | Cropovidone | | | |
| | | Lactose | | | |
| | | anhydrous | | | |
| New | Injectable | Everolimus | 958.2 | 2.4 | 6.89% |
| Evero- | solution | CS-20 | 1836 | 63.4 | |
| limus | | Saline | | | |

### Example 8

### In Vitro Release Test for Paclitaxel-Containing Polymeric Micelle

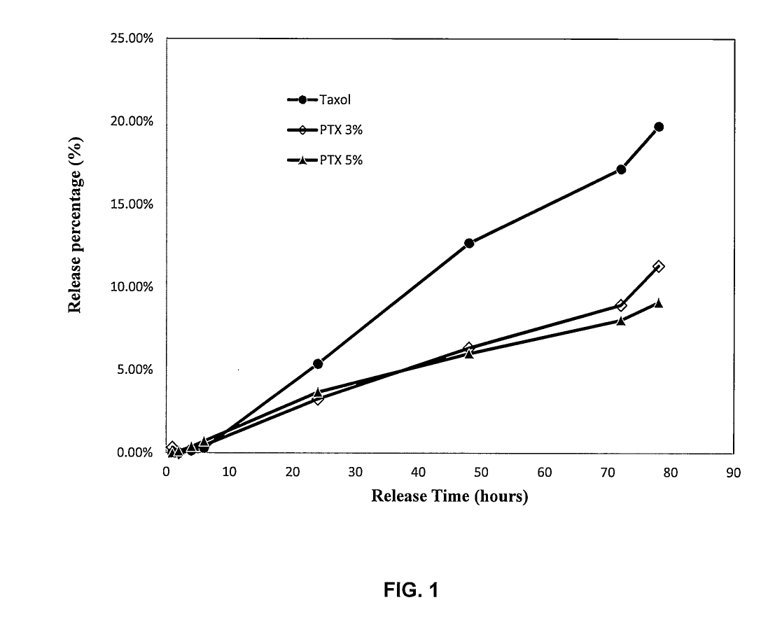

In vitro release tests were conducted with polymeric micelle samples containing paclitaxel. Four samples prepared with paclitaxel and CS-20 were used for in vitro release test. Due to the limited solubility of paclitaxel in plain water, 1.0 M sodium salicylate was used as medium for release test. The test was conducted with 1.0 mL of dialysis tube from spectrum laboratory (FLOAT-A-LYZER G2, CE, 8-10 KD). 0.5 mL of rehydrated polymeric micelle was added to 0.5 mL saline and transferred to the dialysis tube. The dialysis tube was dipped into 15 mL of 1.0 M sodium salicylate in Mill Q water. 200 μl of released samples were pulled from the release medium at the designated time points and then recharged with 200 μl of 1.0 M sodium salicylate solution. The in vitro release results were plotted with the HPLC analytical data (see FIG. 1).

The results show that the polymeric micelle formulations with 3 or 5 wt % of paclitaxel encapsulated exhibited a slower release profile than the commercial Taxol® formulation.

### Example 9

### In Vitro Release Test for Docetaxel-Containing Polymeric Micelle

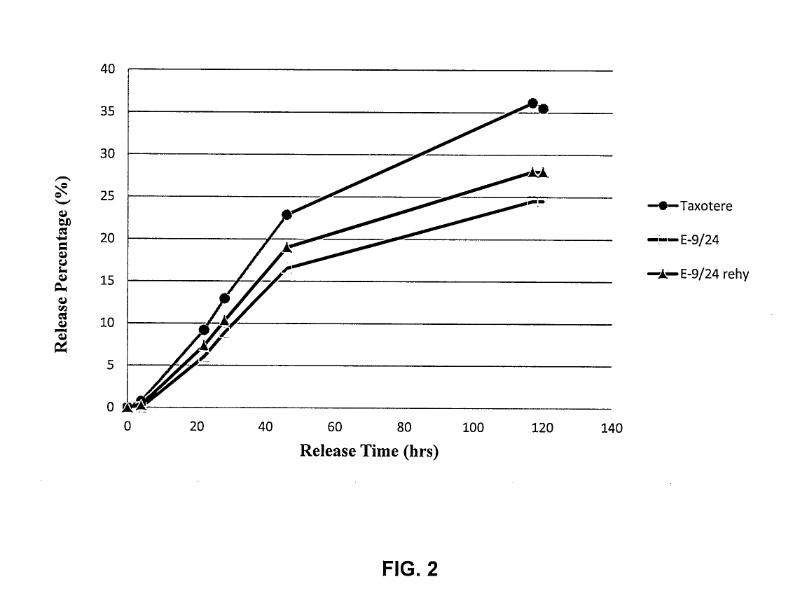

In vitro release tests were also conducted with polymeric docetaxel micelle formulations. Two formulations were prepared with docetaxel and CS-20 and one commercial Taxotere formulation were used for the in vitro release test. Similar to Example 8, 1.0 M sodium salicylate was used as medium for the release test. The test was conducted with 1.0 mL of dialysis tube from spectrum laboratory (FLOAT-A-LYZER G2, CE, 8-10 kD). 0.5 mL of rehydrated polymeric micelle was added to 0.5 mL saline and transferred to the dialysis tube. The dialysis tube was dipped into 15 mL of 1.0 M sodium salicylate in Mill Q water. 200 μl of released samples were pulled from the release medium at the designated time points and then recharged with 200 μl of 1.0 M sodium salicylate solution. The in vitro release results were plotted with the HPLC analytical data (see FIG. 2).

The results show that the polymeric micelle formulations with 5% of docetaxel encapsulated before and after reconstitution exhibited a slower release profile than the commercial Taxotere® formulation.

### Example 10

### In Vitro Cell Killing Study

The in vitro cell killing study was conducted with the following four formulations: Taxotere®, polymeric micelle paclitaxel, polymeric micelle docetaxel, and the solution of CS-20 against the non-small lung cancer cell line of A549. Alarm blue assay was utilized to evaluate the four formulations by comparing their 1050 after 2-hour and 72-hour treatment. The results were summarized in Table 10.

TABLE 10
IC₅₀ of Experimental Formulations against A549 Cell Line

| Sample ID | 2 hr IC₅₀ (μM) | 72 hr IC₅₀ (μM) |
|---|---|---|
| CS-20 | >10 | 9.06 |
| Taxotere ® | 0.019 | <0.0015 |
| New Paclitaxel | 0.126 | 0.009 |
| New Docetaxel | 0.023 | 0.002 |

The results show that after 2-hour treatment, no detectable cytotoxicity was observed for the blank polymeric micelles. The commercial Taxotere® exhibited the lowest IC₅₀ of 0.019 μM compared to both the polymeric micelle docetaxel formulation, which suggests that the polymeric micelle formulations of the present invention are less toxic than Taxotere®. Similar results were shown after 72-hour treatment. Compared to the docetaxel formulation, the polymeric micelle paclitaxel formulation was less toxic which should attribute to the differences in toxicity of the two molecules.

Having illustrated and described the principles of the present invention, it should be apparent to persons skilled in the art that the invention can be modified in arrangement and detail without departing from such principles. Those skilled in the art will recognize, or be able to ascertain using no more than routine experimentation, many equivalents to the specific embodiments of the invention described herein. Such equivalents are intended to be encompassed by the following claims.

All publications and published patent documents cited in this specification are incorporated herein by reference to the same extent as if each individual publication or patent application was specifically and individually indicated to be incorporated by reference.

### REFERENCES

[1] A. A. Date, M. S. Nagarsenker. Parenteral microemulsions: An overview. International Journal of Pharmaceutics, 355 (2008) 19-30.
[2] Taxol® (paclitaxel) for injection, package insert, Bristol-Myers Squibb Company, Princeton, N.J. 08543 USA, Rev April 2011.
[3] Ixempra® Kit (ixabepilone) for injection, package insert, Bristol-Myers Squibb Company, Princeton, N.J. 08543 USA, Rev October 2011.
[4] Sandimmune® (cyclosphorine) for injection, package insert, Novartis Pharmaceuticals Corporation, East Hanover, N.J. 07936, July 2011.
[5] Vumon® (teniposide) for injection, package insert, Bristol-Myers Squibb Company, Princeton, N.J. 08543 USA, Rev October 2011.
[6] Taxotere® (docetaxol) for injection concentrate, package insert, Sanofi-Avantis U.S. LLC, Bridgewater, N.J. 08807, U.S., 2010.
[7] Toposar® (etoposide) for injection, package insert, Pharmacia & Upjohn Company, Kalamazoo, Mich. 49001, USA, Revised December 1998.
[8] H. Gelderblom, J. Verweij, K. Nooter, A. Sparreboom. Cremophor EL: the drawbacks and advantages of vehicle selection for drug formulation. European Journal of Cancer 37 (2001) 1590-1598.
[9] Sang-chul Kim, et al. Biodegradable polymeric micelle-type composition and method for the preparation thereof. U.S. Pat. No. 6,322,805 B1, Nov. 27, 2001.
[10] Shimizu, et al., Micellar preparation containing sparingly water-soluble anticancer agent and novel block copolymer. U.S. Pat. No. 7,820,759, Oct. 26, 2010.
[11] C. Bordes, et al., Determination of poly(_-caprolactone) solubility parameters: application to solvent substitution in a microencapsulation process. International Journal of Pharmaceutics 383 (2010) 236-243.
[12] M. Kanjiro, R. J. Christie, K. Kazunori. Polymeric micelles for nano-scale drug delivery. Reactive & Functional Polymers 71 (2011) 227-234.

We claim:

1. A composition comprising polymeric micelles containing a drug with low water solubility, wherein said polymeric micelles comprise a hydrophobic portion formed by sterol rings of pegylated sterols and the drug; and a hydrophilic portion formed by polyethylene glycol chains of the pegylated sterols.

2. The composition of claim 1, comprising from about 60% to about 99.99% by molar percent of said polymeric micelles and from about 0.01% to about 40% by molar percent of said drug.

3. The composition of claim 1, wherein said sterol is an animal, plant, fungal, and/or algal sterol.

4. The composition of claim 1, wherein said pegylated sterols comprise polyethylene glycol molecules with an average molecular weight in the range of 1000 to 5000 daltons.

5. The composition of claim 1, wherein said pegylated sterols are selected from the group consisting of pegylated cholesterol, pegylated sitosterol, pegylated campesterol, pegylated stigmasterol, pegylated campestanol, pegylated brassicasterol, pegylated lathosterol, pegylated ergosterol, pegylated cycloartenol, pegylated cyclolaudenol, pegylated protothecasterol, pegylated 4a-methylergostanol, pegylated 4a-methylclionastanol, pegylated clionastanol, pegylated 24-beta-ethylcholesta-8,22-enol, derivatives thereof, and a combination thereof.

6. The composition of claim 1, wherein said drug comprises a hydrophobic ring and/or an aromatic ring structure selected from the group consisting of a benzyl ring, a pentadiene ring, a thiophene ring, and a furan ring.

7. The composition of claim 1, wherein said drug is selected from the group consisting of an anticancer drug, an immune-suppressant, an anti-fungal drug, an anti-viral drug, an anti-bacterial drug, a tyrosine kinase inhibitor, an antiphlogistic anodyne, a hormone composition, an anti-allergy drug, a hepatism drug, a metabolic drug, a drug for treating a central nervous system disease, a drug for treating a respiratory disease, a drug for treating a peripheral disease, a drug for treating a digestive disease, a drug for treating an infectious disease, and a drug for treating a circulatory disease.

8. The composition of claim 1, wherein said drug is selected from the group consisting of paclitaxel, docetaxel, cyclosporine, teniposide, etoposide, doxorubicin, daunomycin, mitomycin C, sirolimus, everolimus, indomethacin, ibuprofen, lapatinib ditosylate, and biphenyl dimethyl dicarboxylate.

9. The composition of claim 1, further comprising an excipient selected from the group consisting of a diluent, a binder, a disintegrant, a lubricant, and a colorant.

10. A method of preparing a pharmaceutical or veterinary composition comprising polymeric micelles containing a drug with low water solubility, said method comprising:
(a) mixing said drug with at least one pegylated sterol, or derivative thereof; and
(b) subjecting the mixture to solvent evaporation, dialysis, ultrasonification, stirring, or any combination thereof to form polymeric micelles containing the drug, thereby preparing the pharmaceutical or veterinary composition.

11. The method of claim 10, further comprising lyophilizing the mixture to obtain powders.

12. The method of claim 10, wherein said pharmaceutical composition comprises from about 60% to about 99.99% by molar percent of said polymeric micelles and from about 0.01% to about 40% by molar percent of said drug.

13. The method of claim 10, wherein said sterol is an animal, plant, fungal, and/or algal sterol.

14. The method of claim 10, wherein said polymeric micelles comprise pegylated cholesterol, pegylated sitosterol, pegylated campesterol, pegylated stigmasterol, pegylated campestanol, pegylated brassicasterol, pegylated lathosterol, pegylated ergosterol, pegylated cycloartenol, pegylated cyclolaudenol, pegylated protothecasterol, pegylated 4a-methylergostanol, pegylated 4a-methylclionastanol, pegylated clionastanol, pegylated 24-beta-ethylcholesta-8,22-enol, derivatives thereof, or combinations thereof

15. The method of claim 10, wherein said pegylated sterol comprises polyethylene glycol with an average molecular weight in the range of 1000 to 5000 daltons.

16. The method of claim 10, wherein said drug comprises a hydrophobic ring and/or an aromatic ring structure selected from the group consisting of a benzyl ring, a pentadiene ring, a thiophene ring, and a furan ring.

17. The method of claim 10, wherein said drug is selected from the group consisting of an anticancer drug, an immune-suppressant, a tyrosine kinase inhibitor, an antiphlogistic anodyne, a hormone composition, an anti-allergy drug, a hepatism drug, a metabolic drug, a drug for treating a central nervous system disease, a drug for treating a respiratory disease, a drug for treating a peripheral disease, a drug for treating a digestive disease, a drug for treating an infectious disease, and a drug for treating a circulatory disease.

18. The method of claim 17, wherein said drug is selected from the group consisting of paclitaxel, docetaxel, cyclosporine, teniposide, etoposide, doxorubicin, daunomycin, mitomycin C, sirolimus, everolimus, indomethacin, ibuprofen, lapatinib ditosylate, and biphenyl dimethyl dicarboxylate.

19. A method for delivering a drug with low water solubility to a subject in need thereof, said method comprising administering the composition of claim 1 to said subject, thereby delivering said drug to said subject.

20. The method of claim 19, wherein said subject suffers from a disease selected from the group consisting of a cancer, an immune system disorder, an allergy, a liver disorder, a metabolic disorder, a central nervous system disease, a respiratory disease, a peripheral disease, a digestive disease, an infectious disease, and a circulatory disease.
